(19) United States
(12) Patent Application Publication
(10) Pub. No.: US 2024/0352075 A1
(43) Pub. Date: Oct. 24, 2024

(54) PACAP1-23 ANALOG GLYCOPEPTIDES AND METHODS FOR PRODUCING AND USING THE SAME

(71) Applicant: Arizona Board of Regents on Behalf of the University of Arizona, Tucson, AZ (US)

(72) Inventors: Robin POLT, Tucson, AZ (US); Parthasaradhi Reddy TANGUTURI, Tucson, AZ (US); Christopher APOSTOL, Tucson, AZ (US); John STREICHER, Tucson, AZ (US); Lajos SZABO, Tucson, AZ (US)

(21) Appl. No.: 18/572,695

(22) PCT Filed: Jun. 24, 2022
(86) PCT No.: PCT/US2022/034944

§ 371 Date: Dec. 20, 2023

Related U.S. Application Data
(60) Provisional application No. 63/214,740, filed on Jun. 24, 2021.

(51) Int. Cl. C07K 14/00; A61K 38/00; A61K 47/54

(52) U.S. Cl. CPC C07K 14/00; A61K 47/549; A61K 38/00

(57) ABSTRACT

Glycopeptides comprising a peptide that is covalently linked to one or more saccharides. The glycopeptides of the invention have a significantly reduced VPAC2 receptor agonist activity compared to a native PACAP₁₋₂₇ or PACAP₁₋₃₈ while maintaining a good PAC1 and/or VPAC1 receptor agonist activity. In particular, the peptide portion of the glycopeptides of the invention has from about 20 to about 25 amino acid residues, and the saccharide moiety portion of the glycopeptides of the present invention comprises from 1 to about 8 carbohydrates. The present invention also discloses methods for using the glycopeptides of the invention in treating various neurodegenerative diseases.

### RELATED APPLICATION

This application claims priority to U.S. Provisional Application 63/214,740 filed on Jun. 24, 2021, the content of which is incorporated herein by reference in its entireties for all purposes.

### STATEMENT REGARDING FEDERALLY FUNDED RESEARCH

This invention was made with government support under Grant No. R01 NS091238 awarded by National Institute of Neurological Disorders and Stroke. The government has certain rights in the invention.

### FIELD OF THE INVENTION

The present invention relates to glycopeptides comprising a peptide that is covalently linked to one or more saccharides. In particular, the peptide portion of the glycopeptides of the invention has from about 20 to about 25 amino acid residues and at least 75% sequence identity to SEQ ID NO: 1. The saccharide moiety portion of the glycopeptides of the present invention comprises from 1 to about 8 carbohydrates. The present invention also relates to using the glycopeptides of the invention in treating various neurodegenerative diseases.

### BACKGROUND

Neurodegenerative disorders continue to negatively impact the health and quality of life of millions of people worldwide. Many current therapeutic strategies involve only symptomatic alleviation, as there is a severe lack of treatments with the potential to halt or reverse disease progression. A plethora of studies have elucidated the molecular pathophysiology of various neurodegenerative diseases, revealing a complex interplay between neuronal apoptosis (oxidative stress, mitochondrial dysfunction, imbalances in ion homeostasis) and the inflammatory response in the brain. Hyperactivation of microglia, the resident macrophages of the brain, results in the continual release of pro-inflammatory cytokines, which induces oxidative stress, mitochondrial dysfunction, imbalances in ion homeostasis, and eventual cell death. It has been shown that these apoptotic events induce further stimulation of hyperactive microglia, establishing a continual cycle of neuronal cell death and neuroinflammation that promotes disease progression. Thus, one of the biggest challenges associated with treating neurodegenerative diseases is effectively addressing both the apoptotic and neuroinflammatory aspects simultaneously.

Endogenous pleiotropic peptide hormones are a representative class of compounds that may be able to address this critical problem. The pituitary adenylate cyclase activating peptide (PACAP) is one such endogenous peptide that has been shown to elicit neuroprotection and anti-inflammatory activity in animal models of Parkinson's Disease (PD), ischemic stroke, Alzheimer's Disease (AD), traumatic brain injury (TBI), and ethanol toxicity. Interestingly, PACAP's primary sequence has been conserved for millions of years across many different species, which implies it regulates critical biological functions. The wide distribution of PACAP and its cognate receptors in different organ systems throughout the body further indicates their important regulatory roles. PACAP exists as 2 different isoforms containing either 27 or 38 amino acid residues. To date, it has been shown PACAP's biological activities are elicited through three class B G-protein coupled receptors (GPCRs) known as PAC1, VPAC1, and VPAC2. Of note, class B GPCRs contain a large extracellular domain thought to be an affinity trap to initially bind their relatively large cognate peptide ligands, making them structurally distinct from most other members of the GPCR family tree.

In general, PACAP's neuroprotective effects are mediated through PAC1, while anti-inflammatory effects are modulated through VPAC1 and VPAC2. PACAP binds and activates PAC1, VPAC1, and VPAC2 with equally high affinity whereas the vasoactive intestinal peptide (VIP), a structurally homologous relative of PACAP, exhibits high affinity for VPAC1 and VPAC2, but is not selective for PAC1. This difference in affinity is intriguing considering there is striking structural similarity between VIP and PACAP₁₋₂₇. However, positions 4 and 5 in PACAP's and VIP's primary sequences may be the key to fine-tuning receptor selectivity. Positions 4 and 5 in PACAP are occupied by Gly and Ile, respectively, whereas the corresponding residues in VIP are Ala and Val. Gly is a known β-turn inducer while Ala is known to promote α-helical conformations, and Ile and Val have subtly different steric profiles. Thus, it is believed that amino acid substitutions in the “hinge region” can produce analogues with more diverse selectivity profiles.

Because PACAP has a relatively non-selective receptor binding profile, there have been efforts to identify and produce PACAP analogues having PAC1 receptor binding selectivity. While a little success has been attained, currently there is no known compound having a high PAC1 selectivity.

Several structure-activity relationship (SAR) studies have identified that: (i) the minimum sequence required to maintain adequate receptor binding is PACAP₁₋₂₃, and (ii) the 1^{st} 6 residues are required to maintain agonist activity. Due to the weak receptor binding profile of PACAP₁₋₂₃, it was rarely investigated further for its neuroprotective potential. However, it has been demonstrated that PACAP₁₋₂₃ is capable of attenuating MPP⁺-induced apoptosis, mitochondrial dysfunction, and glutamate-induced excitotoxicity despite drastically reduced binding affinity at PAC1. In addition, PACAP₁₋₂₃ was shown to have a relatively comparable potency of PACAP₁₋₃₈ in activating specific downstream signaling pathways.

Both PACAP₁₋₂₇ and PACAP₁₋₃₈ activate VPAC1 and VPAC2 receptors in addition to PAC1. To reduce the undesired side-effects, it may be desirable to pursue PACAP analogs having selective PAC1 receptor agonist activity. In addition, PACAP₁₋₂₇ and PACAP₁₋₃₈ are relatively long in length and difficult to synthesize. Furthermore, due to a relatively long amino acid length of PACAP₁₋₂₇ and PACAP₁₋₃₈, the resulting yield and/or purity are relatively low compared to shorter amino acid peptides.

### SUMMARY

Some aspects of the invention are based inter alia on the observation by the present inventors that (i) shorter peptides are easier to prepare in higher purity compared to longer peptides such as PACAP₁₋₂₇ or PACAP₁₋₃₈ and (2) peptides can be modified to provide a selective PAC1 receptor agonist activity without some of the undesired side-effects observed for PACAP₁₋₂₇ or PACAP₁₋₃₈.

One particular aspect of the invention provides a glycopeptide having from 20 to 25 amino acid residues and at least 75% sequence identity to SEQ ID NO: 1:

(SEQ ID NO: 1)
HSDX¹IFTDSYSRYRKQX²AVKKYLX³X⁴

where
X¹ is glycine, alanine, sarcosine, valine, leucine, isoleucine, alpha-amino-isobutyric acid, or other aliphatic amino acids;
X² is norvaline, methionine, leucine, isoleucine, alpha-amino-isobutyric acid, or other aliphatic amino acids;
X³ is serine, threonine, cysteine, or an allo-threonine or a related compound, e.g. [Palian & Polt J. Org. Chem. 2001, 66, 7178-7183]; and
X⁴ can be absent or is serine, threonine, cysteine, or an allo-threonine or a related compound optionally bearing a second glycoside,
   and wherein said glycopeptide comprises from 1 to about 8 covalently linked carbohydrates.

In some embodiments, at least one serine of the disclosed glycopeptide is glycosylated. In other embodiments, the C-terminus end of the amino acid chain comprises two glycosylated amino acids. Still in other embodiments, at least one amino acid residue is a (D)-isomer.

Yet in other embodiments, X³ is glycosylated with a mono- or a di-saccharide.

In certain embodiments, X⁴ is absent. Still in other embodiments, X⁴ is (L)-serine, (D)-serine, (L)-threonine, (D)-theronine. (L)-cysteine, or (D)-cysteine. Yet in other embodiments, X⁴ is glycosylated with a mono- or a di-saccharide.

In further embodiments, said glycopeptide is a PAC1 agonist. In some instances, PAC1 effective concentration (EC₅₀) of said glycopeptide is about 50 nM or less, typically 25 nM or less, often 10 nM or less, and most often 5 nM or less.

Still in other embodiments, said glycopeptide has VPAC1 receptor binding profile similar to native PACAP₁₋₃₈. In one particular embodiment, VPAC1 effective concentration (EC₅₀) of said glycopeptide is about 200 nM or lower, typically 100 nM or lower, often 75 nM or lower, and more often 50 nM or lower.

In certain embodiments, said glycopeptide has a much lower binding affinity to VPAC2 compared to a native PACAP₁₋₂₇. In some instances, said glycopeptide has at least about 2×, typically at least about 5×, and often at least about 10× lower binding affinity to VPAC2 compared to the native PACAP₁₋₂₇. In one particular embodiment, VPAC2 effective concentration (EC₅₀) of said glycopeptide is about 500 nM or higher, typically 1 μM or higher, and often 5 μM or higher.

Still in other embodiments, glycopeptides of SEQ ID NO: 1 have PAC1 functional activity that is at least about 75%, typically at least about 80%, often at least about 90%, and most often at least 95% compared to the functional activity of PACAP₁₋₂₇. In other embodiments, glycopeptides of SEQ ID NO: 1 have VPAC1 functional activity that is at least about 50%, typically at least about 75%, often at least about 80%, and most often at least 90% compared to the functional activity of PACAP₁₋₂₇. Yet in other embodiments, glycopeptides of SEQ ID NO: 1 have VPAC2 functional activity that is at about 50% or less, typically about 25% or less, often about 10% or less, and most often about 5% or less compared to the functional activity of PACAP₁₋₂₇.

Yet in other embodiments, the ratio of selectivity of PAC1 vs. VPAC1 in glycopeptides of the invention is about 10:1 or greater, typically about 25:1 or greater, often about 50:1 or greater, and most often about 60:1 or greater. In other embodiments, the ratio of selectivity of PAC1 vs. VPAC2 in glycopeptides of the invention is about 25:1 or greater, typically about 50:1 or greater, often about 75:1 or greater, and most often about 100:1 or greater.

Yet in other embodiments, said saccharide comprises from 1 to 8 carbohydrates. In some instances, said saccharide is a monosaccharide, a disaccharide, or a combination thereof. Still in other embodiments, said peptide comprises a plurality of glycosylated amino acid residues.

In some embodiments, said saccharide is selected from the group consisting of glucose, maltose, lactose, melibiose, maltotriose, altrose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides.

Another aspect of the invention provides a method for treating a neurodegenerative disease in a subject, said method comprising administering to the subject in need of such a treatment a therapeutically effective amount of a glycopeptide disclosed herein.

In some embodiments, said neurodegenerative disease is selected from the group consisting of amyotrophic lateral sclerosis. Parkinson's Disease, migraine attacks, traumatic brain injury, stroke, and dementia.

### BRIEF DESCRIPTION OF THE DRAWINGS

The features and advantages of the present invention will become apparent from a consideration of the following detailed description presented in connection with the accompanying drawings in which:

FIGS. 1A-1B show non-limiting examples of strategic amino acid substitutions to fine-tune receptor selectivity and optimize stability and BBB transport. The bioactive conformation of PACAP analogues can be fine-tuned by introducing amino acids into the 4th position that are biased towards either α-helical or β-turn-like conformations.

FIG. 2 shows amino acid building blocks utilized to suppress aspartimide formation during synthesis of the glycopeptides.

FIGS. 3A-3B show the general synthetic scheme for the glycopeptide preparation and the materials and coupling protocols utilized in the preparation. The glycopeptides were constructed on a Rink Amide HMBA resin, and coupling protocols using DIC/6-Cl-HOBt or HBTU/NMM were utilized depending on the identity of the amino acids (FIG. 3A). The acetate protecting groups in the glycoside moieties were cleanly removed using 50% H₂N—NH₂·H₂O in NMP. Global side chain deprotection and resin cleavage were carried out using a cocktail consisting of TFA/Et₃SiH/anisole/DCM/H₂O.

### DETAILED DESCRIPTION

As used in this specification and the appended claims, the singular forms “a,” “an” and “the” include plural references unless the content clearly dictates otherwise.

The terms “saccharide” and “carbohydrate” are used interchangeably herein and refer to aldoses and ketoses consisting of carbon (C), hydrogen (H) and oxygen (O) atoms, typically, but not necessarily, with a hydrogen-oxygen atom ratio of 2:1. The term also includes mono-deoxy-carbohydrates, such as deoxyribose, etc. where one hydroxy group is removed from the empirical formula Cₘ(H₂O)ₙ formula, where m is typically 6 and n can be 5 or 6.

The terms “sugar” refers to a mono- and/or disaccharide.

The term “monosaccharide” refers to any type of hexose of the formula C₆H₁₂O₆ or a derivative thereof. The ring structure (i.e., ring type) of the monosaccharide can be a pyranose or a furanose. In addition, the monosaccharides can be an α- or β-anomer. Monosaccharide can be a ketonic monosaccharide (i e, ketose), an aldehyde monosaccharide (i.e., aldose), or any type of hexose of the formula C₆H₁₂O₆ or a derivative thereof. Exemplary aldoses of the invention include, but are not limited to, allose, altrose, glucose, mannose, gulose, idose, galactose, talose, ribose, arabinose, xylose, lyxose, and derivatives thereof. Exemplary ketoses of the invention include, but are not limited to, psicose, fructose, sorbose, tagatose, ribulose, xylulose, and derivatives thereof. As used herein the term “derivative” refers to a derivative of a monosaccharide in which one or more of the hydroxyl groups is replaced with hydrogen (e.g., 2-deoxy glucose, 5-deoxyglucose, etc.), an amine (e.g., amino sugars) or is replaced with a halogen, such as chloro, fluoro or iodo. (e.g., 5-fluoroglucose, 2-fluoroglucose, 5-chrologlucose, 2-chloroglucose, etc.). Monosaccharide can be an (L)-isomer or a (D)-isomer.

The term “disaccharide” refers to a carbohydrate composed of two monosaccharides. It is formed when two monosaccharides are covalently linked to form a dimer. The linkage can be a (1→4) bond, a (1→6) bond, a (1→2) bond, etc. between the two monosaccharides. In addition, each of the monosaccharides can be independently an α- or β-anomer. Exemplary disaccharides that can be used in the present invention include, but are not limited to, sucrose, lactose, altose, maltose, trehalose, cellobiose, lactulose, and chitobiose, etc. Each of the monosaccharides can independently be a ketonic monosaccharide (i.e., ketose), an aldehyde monosaccharide (i.e., aldose), or any type of hexose of the formula C₆H₁₂O₆ or a derivative thereof. Exemplary aldoses that can be used in preparing disaccharides of the invention include, but are not limited to, allose, altrose, glucose, mannose, gulose, idose, galactose, talose, ribose, arabinose, xylose, lyxose, and derivatives thereof. Exemplary ketoses that can be used in preparing disaccharides of the invention include, but are not limited to, psicose, fructose, sorbose, tagatose, ribulose, xylulose, and derivatives thereof. Each monosaccharide can also be independently an (L)-isomer or a (D)-isomer.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

As used herein, the term “treating”, “contacting” or “reacting” when referring to a synthesis or chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The terms “identical,” “identity,” “percent identity,” “percent sequence identity,” and “sequence identity” are used interchangeably herein. In particular, in the context of comparison of two or more peptides, these terms refer to two or more sequences or subsequences that are the same or have a specified percentage of nucleotides or amino acid residues that are the same, when compared and aligned for maximum correspondence, as measured using a sequence comparison algorithm known to one skilled in the art, or by visual inspection. For example, 75% sequence identity of a peptide A compared to peptide B means. 75% of the amino acid sequences in peptide A are the same as that of the amino acid sequences of peptide B. The term also includes insertion/addition or deletion of amino acids compared to a reference peptide. Thus, 75% sequence identity of peptide A compared to peptide B can also mean that peptide A has 25% more or 25% less (i.e., +25%) amount of amino acid residues. In particular, if peptide B has 27 amino acids, then 75% sequence identify of peptide A means peptide A can have from 21 to about 33 amino acid residues. In some embodiments, these terms are used to denote sequences which when aligned have similar (identical or conservatively replaced) amino acids in like positions or regions, where identical or conservatively replaced amino acids are those which do not alter the activity or function of the protein as compared to the starting protein. Percent sequence identity may be calculated by determining the number of residues that differ between a peptide encompassed by the present invention and a reference peptide such as SEQ ID NO: 1, taking that number and dividing it by the number of amino acids in the reference peptide, multiplying the result by 100, and subtracting that resulting number from 100. For example, a sequence having 35 amino acids with four amino acids that are different would have a percent (%) sequence identity of 89% (e.g. 100−((4/35)×100)). For a peptide having a sequence that is longer than the number of amino acids in a reference peptide, the number of residues that differ from the reference peptide will include the additional (or difference in) amino acids over (or under) 35 for purposes of the aforementioned calculation. For example, a sequence having 37 amino acids, with four amino acids different from the 35 amino acids in the reference peptide sequence and with two additional amino acids at the carboxy terminus which are not present in the reference peptide sequence, would have a total of six amino acids that differ from the reference peptide. Thus, this sequence would have a percent (%) sequence identity of 83% (e.g. 100−((6/35)×100)). The degree of sequence identity may be determined using methods well known in the art (sec, for example, Wilbur, W. J. et al., Proc. Natl. Acad. Science USA, 1983, 80, 726-730 and Myers E. et al., Comput. Appl. Biosci., 1988, 4, 11-17. One program which may be used in determining the degree of similarity is the MegAlign Lipman-Pearson one pair method (using default parameters) which can be obtained from DNAstar Inc, 1128, Selfpark Street, Madison, Wisconsin, 53715, USA as part of the Lasergene system. Another program, which may be used, is Clustal W. This is a multiple sequence alignment package developed by Thompson et al. (Nucleic Acids Research, 1994, 22 (22), 4673-4680) for DNA or protein sequences. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen clearly.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “about” as used herein is not intended to limit the scope of the invention but instead encompass the specified material, parameter, or step as well as those that do not materially affect the basic and novel characteristics of the invention. The term “about” or “approximately” as used herein refers to being within an acceptable error range for the particular value as determined by one of ordinary skill in the art, which will depend in part on how the value is measured or determined. i.e., the limitations of the measurement system, i.e., the degree of precision required for a particular purpose, such as a pharmaceutical formulation. For example, “about” can mean within 1 or more than 1 standard deviation, per the practice in the art. Alternatively, “about” can mean a range of ±20%, typically ±10%, often ±5% and more often ±1% of a given numeric value.

The term “derivative” refers to (i) any chemical modification of the amino acid, such as alkylation (e.g., methylation or ethylation) of the amino group or functional group on the side chain, removal of the side-chain functional group, etc. (e.g., conversion of —OH, —SH—, or —NH₂ to H); and/or (ii) conservative substitutions of amino acid.

As used herein, the term “conservative substitutions of amino acid” refers to replacing an amino acid with another amino acid having a similar side-chain functional group. For example, basic amino acids that can be replaced or substituted by one another include arginine, lysine and histidine. Acidic amino acids that can be replaced or substituted by one another include glutamic acid and aspartic acid. Polar amino acids that can be replaced or substituted by one another include glutamine and asparagine. Hydrophobic amino acids that can be replaced or substituted by one another include leucine, isoleucine, and valine. Aromatic amino acids that can be replaced or substituted by one another include phenylalanine, tryptophan, and tyrosine. Small amino acids that can be replaced or substituted by one another include glycine, alanine, serine, threonine, and methionine. In general, amino acid substitutions that do not generally alter specific activity are known in the art and are described, for example, by H. Neurath and R. L. Hill, 1979, in “The Proteins,” Academic Press, New York. Some of the exemplary common amino acid substitutions include, but are not limited to, Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

The term “retro modified” refers to a peptide which is made up of L-amino acids in which the amino acid residues are assembled in opposite direction to the native peptide with respect the which it is retro modified.

The term “inverso modified” refers to a peptide which is made up of D-amino acids in which the amino acid residues are assembled in the same direction as the native peptide with respect to which it is inverso modified.

The term “retro-inverso modified” refers to a peptide which is made up of D-amino acids in which the amino acid residues are assembled in the opposite direction to the native peptide with respect to which it is retro-inverso modified.

The term “native” refers to any sequence of L amino acids used as a starting sequence or a reference for the preparation of partial or complete retro, inverso or retro-inverso analogues.

The term “peptide” as used throughout the specification and claims is to be understood to include amino acid chain of any length.

Each amino acid of glycopeptides of the invention can be a natural or unnatural amino acid. Natural amino acids are well known to one skilled in the art and refers to proteinogenic amino acids. Unnatural amino acids refer to non-proteinogenic amino acids that either occur naturally or are chemically synthesized. An organic compound with an amine (—NH₂) and a carboxylic acid (—COOH) functional group is an amino acid. The proteinogenic amino acids are small subset of this group that possess central carbon atom (α- or 2-) bearing an amino group, a carboxyl group, a side chain and an α-hydrogen levo conformation, with the exception of glycine, which is achiral, and proline, whose amine group is a secondary amine and is consequently frequently referred to as an imino acid for traditional reasons, albeit not an imino. The genetic code encodes 20 standard amino acids for incorporation into proteins during translation. Two extra proteinogenic amino acids: selenocysteine and pyrrolysine, do not have a dedicated codon, but are added in place of a stop codon when a specific sequence is present, UGA codon and SECIS element for selenocysteine, UAG PYLIS downstream sequence for pyrrolysine. All other amino acids are termed “non-proteinogenic”.

One particular aspect of the invention provides a glycopeptide. A glycolpeptide refers to a peptide that is covalently linked to one or more saccharides, i.e., a glycosylated peptide. It should also be appreciated that the scope of the invention includes peptides that are retro modified, inverso modified, and retro-inverso modified glycopeptides. In one particular embodiment, the glycopeptide of the invention comprises a peptide that is covalently linked to a saccharide. The peptide consists of from 20 to 25 amino acid residues and has at least 75% sequence identity to SEQ ID NO:1:

(SEQ ID NO: 1)
HSDX¹IFTDSYSRYRKQX²AVKKYLX³X⁴

where
X¹ is glycine, alanine, sarcosine, valine, leucine, isoleucine, alpha-amino-isobutyric acid, or other aliphatic amino acids;
X² is norvaline, methionine, leucine, isoleucine, alpha-amino-isobutyric acid, or other aliphatic amino acids;
X³ is serine, threonine, cysteine, or an allo-threonine or a related compound, e.g. [Palian & Polt J. Org. Chem. 2001, 66, 7178-7183]; and
X⁴ can be absent or is serine, threonine, cysteine, or an allo-threonine or a related compound,
   and wherein said glycopeptide comprises from 1 to about 8 covalently linked carbohydrates.

Typically, the side-chain functional group of an amino acid is glycosylated. In one particular embodiment, at least one serine is glycosylated. In another embodiment, at least two amino acid residues in the peptide are glycosylated. In one particular embodiment, the C-terminus end of said peptide comprises two glycosylated amino acids. It should be appreciated that in SEQ ID NO:1, each amino acid residue can independently be an (L)-isomer or a (D)-isomer.

Without being bound by any theory, it is generally believed that inter alia (i) PAC1 receptor generally affects apoptosis inside the CNS; (ii) VPAC1 affects dilation of the capillaries outside the CNS and increases blood flow to the affected brain region; and (iii) VPAC2 affects recruitment of leukocytes and other immune cells, and leads to infiltration and edema (i.e., brain swelling).

Surprisingly and unexpectedly, the present inventors have discovered that glycopeptides of the invention substantially retain or even improves (i.e., having at least 70%, typically at least 80%, often at least about 90%, and most often 100% or more) PAC1 and VPAC1 receptor activity profiles of native PACAP₁₋₃₈ while having substantially reduced (i.e., having about 1.5 times less, typically having at least about 2× or less, often having at least 5× or less, and more often having at least 10× or less) VPAC2 receptor activity profile of native PACAP₁₋₃₈. Thus, some glycopeptides of the invention have the positive benefits of PAC1 and VPAC1 receptor activation, and in some instances even provides synergistic effect by simultaneously activating both of these receptors centrally and peripherally. Again, without being bound by any theory, it is believed that the presence of the saccharide completely eliminate or significantly reduce activation of VPAC2, which is believed to cause edema. Accordingly, some glycopeptides of the invention have a desired receptor binding profile in the brain, namely, increased blood supply (peripheral effects) and neuroprotection (central effects).

In some embodiments, at least one amino acid residue is glycosylated, i.e., covalently linked to a saccharide. Still in other embodiments, at least two amino acid residues are glycosylated.

The glycopeptides disclosed here are stable and can cross the blood-brain barrier (BBB). Glycopeptides are peptides that contain carbohydrate moieties (glycans or saccharides) covalently attached to the side chains of the amino acid residues that constitute the peptide. In particular, glycopeptides of the invention include a peptide that is covalently linked to a saccharide. The peptide portion of the glycopeptide of the invention has from about 20 to about 25 amino acid residues and at least 75% sequence identity to SEQ ID NO: 1. The saccharide portion of the glycopeptide of the invention ranges from 1 to about 8 carbohydrates.

Another aspect of the invention provides a method for treating amyotrophic lateral sclerosis. Huntington's Disease, Parkinson's Disease. Alzheimer's Disease, traumatic brain injury, and other neurodegenerative diseases. In one particular aspect of the invention, a method for treating a neurodegenerative disease is provided. The method includes administering to the subject in need of such a treatment a therapeutically effective amount of a glycopeptide of the invention.

This disclosure can be further illustrated by the following Items:

1. A composition comprising a glycopeptide, said glycopeptide comprising a peptide that is covalently linked to a saccharide, wherein said peptide consists of from 20 to 25 amino acid residues and at least 75% sequence identity to SEQ ID NO: 1:

(SEQ ID NO: 1)
HSDX¹IFTDSYSRYRKQX²AVKKYLX³X⁴

wherein
X¹ is glycine, alanine, sarcosine, valine, leucine, isoleucine, alpha-amino-isobutyric acid, or other aliphatic amino acids;
X² is norvaline, methionine, leucine, isoleucine, alpha-amino-isobutyric acid, or other aliphatic amino acids;
X³ is serine, threonine, cysteine, or an allo-threonine or a related compound; and
X⁴ can be absent or is serine, threonine, cysteine, or an allo-threonine or a related compound,
   and wherein said glycopeptide comprises from 1 to 8 covalently linked carbohydrates.

2. The composition of Item 1, wherein at least one serine is glycosylated.

3. The composition of any preceding Items, wherein the C-terminus end of said peptide comprises two glycosylated amino acids.

4 The composition of any preceding Items, wherein at least one amino acid residue is a (D)-isomer.

5. The composition of any preceding Items, wherein X² is norvaline.

6. The composition of any preceding Items, wherein X³ is glycosylated with a mono- or a di-saccharide.

7. The composition of any preceding Items, wherein the sequence of the peptide is selected from the group consisting of SEQ ID Nos: 2-7.

8. The composition of any preceding Items, wherein X⁴ is (L)-serine, (D)-serine, (L)-threonine, (D)-theronine, (L)-cysteine, or (D)-cysteine.

9. The composition of any preceding Items, wherein X⁴ is glycosylated with a mono- or a di-saccharide.

5. The composition of any preceding Items, wherein said glycopeptide is a PAC1 agonist.

6 The composition of any preceding Items, wherein PAC1 effective concentration (EC₅₀) of said glycopeptide is about 10 nM or less.

7. The composition of any preceding Items, wherein said glycopeptide shows additional binding and activation toward VPAC1

8. The composition of any preceding Items, wherein VPAC1 effective concentration (EC₅₀) of said glycopeptide is about 100 nM or lower.

9. The composition of any preceding Items, wherein said glycopeptide has a much lower binding affinity to VPAC2 compared to binding affinity to VPAC2 by a native PACAP₁₋₂₇.

10. The composition of any preceding Items, wherein said glycopeptide has at least 10× lower binding affinity to VPAC2 compared to said native PACAP₁-27.

11. The composition of any preceding Items, wherein VPAC2 effective concentration (EC₅₀) of said glycopeptide is about 1 μM or greater.

12. The composition of any preceding Items, wherein said saccharide comprises from 1 to 8 carbohydrates.

13 The composition of any preceding Items, wherein said saccharide is a monosaccharide, a disaccharide, or a combination thereof.

14. The composition of any preceding Items, wherein said peptide comprises a plurality of glycosylated amino acid residues.

15. The composition of any preceding Items, wherein said saccharide is selected from the group consisting of glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides.

16. A method for treating a neurodegenerative disease in a subject, said method comprising administering to the subject in need of such a treatment a therapeutically effective amount of a glycopeptide of of any preceding Items.

17. The method of Item 21, wherein said neurodegenerative disease is selected from the group consisting of amyotrophic lateral sclerosis. Huntington's Disease, Parkinson's Disease, migraine attacks, traumatic brain injury, stroke, and dementia.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Synthesis

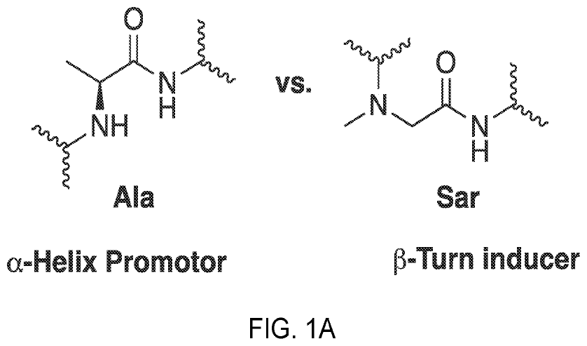

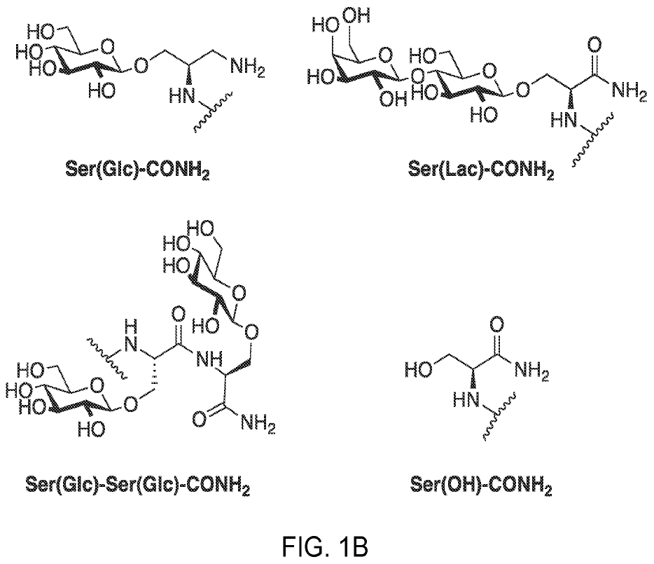

It was believed that implementing glycosylation strategy to produce glycopeptides of SEQ ID NO: 1 would yield glycopeptides with greatly enhanced stability and membrane permeability while still retaining the potency and efficacy similar to native PACAP. This Example illustrates synthesis and in vitro characterization of glycopeptides of SEQ ID NO: 1 containing various carbohydrate motifs (glucose, di-glucose, lactose, FIG. 1A) to assess their effects on in vitro potency and efficacy and additional substitutions at the N-terminus that fine-tune receptor selectivity. In one example. Ala, a known α-helix promoter, and N-methylglycine (Sar) (FIG. 1A), which has been shown to induce β-turn-like conformations, were introduced in order to fine-tune the receptor selectivity.

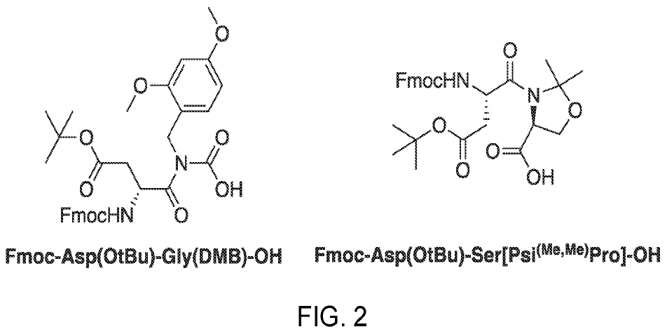

The glycopeptides of SEQ ID NO: 1 were synthesized on a rinke amide MBHA resin (0.25 mmol scale, d.s. ˜0.5 mmol/g) using the Prelude® automated peptide synthesizer from Gyros Protein Technologies. The carbohydrate motifs were introduced as pre-assembled Fmoc protected serine glycoside buildings blocks, which were prepared utilizing minimally competent InBr; catalysis. See, for example, Lefever et al., Carbohydr. Res. 2012, 357, 121-125. doi: 10.1016/j.carres.2012.01.008. Several different coupling protocols were utilized in this synthesis. SEQ ID NO: 1's relatively long length necessitated the use of stronger coupling reagents later on in the synthesis, and there are two motifs present that are highly prone to aspartimide formation (Asp³-Gly⁴ and Asp⁸-Ser⁹). Aspartimide formation involves the base-promoted cyclization of an aspartic acid side chain with the α-amino nitrogen of the preceding residue, with Gly, Ser. Thr, and Cys being the most problematic. To circumvent this issue, dipeptide building blocks that suppress aspartimide formation were used. In the case of the Asp⁸-Ser⁹ motif, a pseudoproline dipeptide building block (Fmoc-DS, FIG. 2) was utilized, and for the Asp³-Gly⁴, a dimethoxybenzyl (DMB)-containing dipeptide (Fmoc-DG, FIG. 2) was utilized.

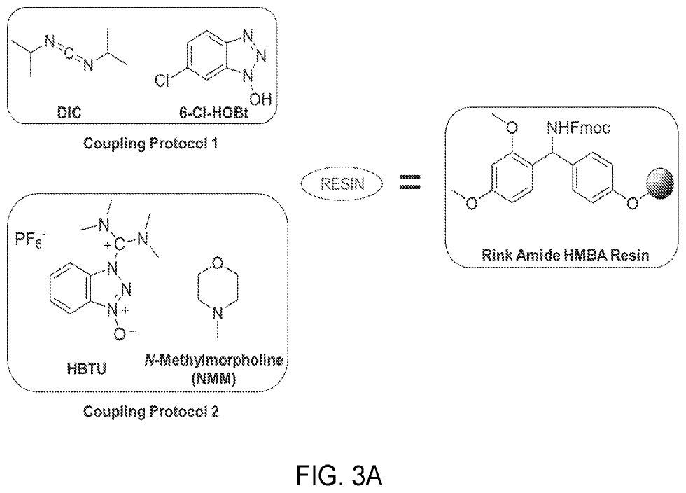

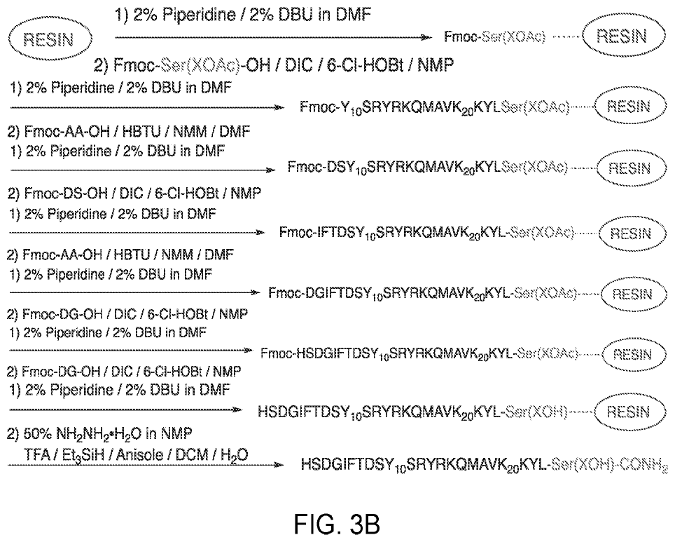

With this plan in hand, synthesis of glycopeptides of SEQ ID NO:1 was carried out. First, the desired Fmoc protected glycosyl amino acid or Fmoc-Ser (OtBu)-OH were initially loaded onto the resin using equimolar amounts of Cl-HOBt and DIC in NMP (FIGS. 3A-3B).

The resin was then treated with a mixture of DIPEA/Ac₂O in DCM (10%/10% v/v) to cap any unreacted sites on the resin. The next 14 residues (Tyr¹⁰-Leu²³) were coupled utilizing a standard HBTU/N-methylmorpholine coupling protocol (FIG. 3B). The Fmoc-DS dipeptide was coupled using the Cl-HOBt/DIC to introduce Asp⁸ and Ser⁹. The relatively lipophilic tripeptide motif Ile⁵-Phe⁶-Thr⁷ was introduced using a modified HBTU/N-methylmorpholine coupling protocol where a greater excess of the coupling reagent and base were used. The Fmoc-DG dipeptide was utilized in cases where the Asp³-Gly⁴ motif was present. In the cases of CRA_TG 3026 and CRA_TG 3027 (Table 1), the Cl-HOBt/DIC coupling protocol was utilized to couple Ala⁴ or Sar⁴. The remaining amino acids were coupled using the Cl-HOBt/DIC in NMP protocol. After cleavage of the final Fmoc group on His¹, the resin was treated with 50% NH₂NH₂·H₂O in NMP to remove the carbohydrate acetate protecting groups. The crude peptides were precipitated in cold diethyl ether and purified by preparative HPLC. The final purity of glycopeptides of SEQ ID NO:1 was superior to PACAP₁₋₂₇ glycopeptides previously prepared.

TABLE 1
Representative examples (SEQ ID Nos: 2-7)
of glycopeptides of SEQ ID NO: 1

| | | | | | | | Amino Acid |
|---|---|---|---|---|---|---|---|
| | PAC1 | | VPAC1 | | VPAC2 | | Sequence |
| Compound | EC₅₀ (nM) | E_{MAX} (%) | EC₅₀ (nM) | E_{MAX} (%) | EC₅₀ (nM) | E_{MAX} (%) | (Glycoside) |
| PACAP₁₋₂₇ | 20, 9.0 | 100, 100 | 30, 6.5 | 100, 100 | 71, 29 | 100, 100 | [symbol] |
| CRA-DV- | 2.2 | 198 | 5.2 | 42 | 571 | 111 | [symbol] |
| 3022 | | | | | | | |
| CRA-DV- | 0.64 | 181 | 37 | 105 | 567 | 110 | [symbol] |
| 3023 | | | | | | | |
| CRA-DV- | 1.9 | 152 | 26 | 147 | 7369 | 128 | [symbol] |
| 3024 | | | | | | | |
| CRA-DV- | 1.8 | 174 | 21 | 118 | 1943 | 168 | [symbol] |
| 3025 | | | | | | | |
| CRA-DV- | 7 | 181 | 35 | 124 | 321 | 190 | [symbol] |
| 3026 | | | | | | | |
| CRA-DV- | 67 | 134 | 195 | 107 | NC | NC | [symbol] |
| 3027 | | | | | | | |

¹ Ñ = Norvaline, Σ = Sarcosine [symbol]
indicates data missing or illegible when filed

### In Vitro Characterization

TABLE 2
Characterization of Synthesized Glycopeptides of SEQ ID NO: 1

| | | | | | HPLC |
|---|---|---|---|---|---|
| | | | | | Retention |
| Compound | | Mol. Formula | Exact Mass | Mass found¹ | Time (min.) ² |
| CRA_DV_3022 | SEQ ID NO 2 | C₁₂₈H₁₉₉N₃₇O₃₇ | 2846.48 | 570.71 (M + 5H)⁵⁺ | 13.98 |
| CRA_DV_3023 | SEQ ID NO 3 | C₁₃₄H₂₀₉N₃₇O₄₂ | 3008.54 | 603.09 (M + 5H)⁵⁺ | 13.60 |
| CRA_DV_3024 | SEQ ID NO 4 | C₁₄₃H₂₂₄N₃₈O₄₉ | 3257.62 | 652.76 (M + 5H)⁵⁺ | 13.24 |
| CRA_TG_3025 | SEQ ID NO 5 | C₁₄₀H₂₁₉N₃₇O₄₇ | 3170.59 | 793.93 (M + 4H)⁴⁺ | 13.59 |
| CRA_TG_3026 | SEQ ID NO 6 | C₁₄₁H₂₂₁N₃₇O₄₇ | 3184.60 | 638.09 (M + 5H)⁵⁺ | 13.48 |
| CRA_TG_3027 | SEQ ID NO 7 | C₁₄₁H₂₂₁N₃₇O₄₇ | 3184.60 | 638.01 (M + 5H)⁵⁺ | 13.42 |

¹m/z values evaluated by ESI-MS.
² HPLC conditions: 5-80% CH₃CN vs 0.1% CF₃COOH in H₂O over 60 min

TABLE 3
In vitro cAMP stimulation of glycopeptides of SEQ ID NO: 1

| | PAC1 | | VPAC1 | | VPAC2 | |
|---|---|---|---|---|---|---|
| Compound | EC₅₀ (nM) | Eₘₐₓ (%) | EC₅₀ (nM) | Eₘₐₓ (%) | EC₅₀ (nM) | Eₘₐₓ (%) |
| PACAP₁₋₂₇ | 20, 9.0 | 100, 100 | 30, 6.5 | 100, 100 | 71, 29 | 100, 100 |
| CRA-DV- | 2.2 | 198 | 5.2 | 94 | 571 | 91 |
| 3022 | | | | | | |
| CRA-DV- | 0.64 | 181 | 37 | 105 | 567 | 90 |
| 3023 | | | | | | |
| CRA-DV- | 1.9 | 152 | 26 | 127 | 7369 | 130 |
| 3024 | | | | | | |
| CRA-TG- | 1.8 | 174 | 21 | 91 | 1943 | 118 |
| 3025 | | | | | | |
| CRA-TG- | 7.0 | 181 | 38 | 95 | 321 | 115 |
| 3026 | | | | | | |
| CRA-TG- | 67 | 134 | 195 | 90 | NC | NC |
| 3027 | | | | | | |

NC = No Convergence (no activity)

Cell Culture: CHO cells stably expressing cloned PAC1, VPAC1, and VPAC2 were produced by electroporation with human PAC1/VPAC1/VPAC2 N-3×HA tag cDNA constructs (GeneCopocia). Cells were grown on 10 cm dishes in DMEM/F-12 50/50 mix w/L-glutamine & 15 mM HEPES (Corning) containing 10% heat inactivated fetal bovine serum, 100 units/mL penicillin, 100 μg/mL streptomycin, and 500 μg/mL G418 under 5% CO₂ at 37° C. The cells were enriched into high expressing populations using flow cytometry, selecting the top ˜2% of expressing cells.

CAMP Accumulation Assay: At ˜80% confluence, cells were plated into 96-well plates (20.000 cells/well) and grown in the same medium and conditions as described above for 24 hrs. The cells were then serum starved for 4 hr. After a 20 min incubation at 37° C. with 500 μM 3-Isobutyl-1-methylxanthine (IBMX), serum free medium containing 500 μM IBMX and the appropriate agonists were added and then incubated for 10 min at 37° C. The reaction was terminated by removing the medium and adding 60 μL of ice-cold assay buffer (50 mM Tris-HCl pH 7.4, 100 mM NaCl, 5 mM ethylenediaminetetraacetic acid [EDTA]). Plates were sealed with boiling mats and then boiled at 95° C. for 10 min. Plates were then centrifuged at 4000 rpm, 4° C., for 10 min to remove debris. 50 μL of lysate was transferred to a 96-well plate. Lysate was incubated with ˜1 μmol 3H-CAMP (PerkinElmer), and 7 μg protein kinase A (Sigma Aldrich) with 0.05% Bovine Serum Albumin (BSA). The assay was incubated at room temperature for 1 hr. The reactions were then harvested onto GF/B filter plates (PerkinElmer) via rapid filtration by a 96-well plate Cell Harvester (Brandel) and washed 3 times with ice-cold water. Filter plates were dried, 40 μL of Microscint-PS scintillation cocktail was added to each well, and then counted in a TopCount or Microbeta2 (PerkinElmer) microplate scintillation counter.

All derivatives showed strong efficacy at the primary target, PAC1. All compounds also show at least reasonable potency, showing that the glycosylation and other modifications made do not compromise the ability of the ligands to engage the target. Most compounds had low nM or even sub-nM potency, with the exception of CRA-DV-3027, which still had a reasonable 67 nM potency. At the other 2 targets, a consistent loss of potency was seen with VPAC1, increasing even more at VPAC2, consistent with past experience screening these targets. Full efficacy was maintained. The most selective ligand is CRA-DV-3023 with about 60 fold selectivity versus VPAC1 and ˜900 fold selectivity versus VPAC2. These results demonstrate our ability to make modified PACAP ligands that will preferentially engage the primary receptor target while developing selectivity versus the two most closely related off-target receptors, all while maintaining efficacy with expected pharmacokinetic benefits.

Some glycopeptides of SEQ ID NO: 1 were shown to be as potent as native PACAP₁₋₃₈ at attenuating glutamate-induced cytotoxicity, MPP⁺-induced cell death, and maintaining cellular structural integrity, suggesting that glycopeptides of SEQ ID NO: 1 are a viable alternative to PACAP₁₋₂₇ and/or PACAP₁₋₃₈. This is advantageous for several reasons. First, PACAP₁₋₂₇ and PACAP₁₋₃₈ are relatively long in length and difficult to synthesize, but the shorter glycopeptides of SEQ ID NO: 1 are easier to prepare and in higher purity. To this end, a variety of representative glycopeptides of SEQ ID NO: 1 were prepared in satisfactory yields and higher purity than those of PACAP₁₋₂₇ glycopeptide analogues. Glycopeptides of SEQ ID NO: 1 include various glycoside motifs at the C-terminus (e.g., glucose, di-glucose, lactose) to enhance stability and BBB transport and N-terminal substitutions in position 4 (Ala, N-methyl glycine) to fine-tune receptor selectivity.

### Materials and Methods

Glycopeptide Synthesis and Purification: Fmoc-Based solid phase peptide synthesis (SPPS) was used to synthesize some of the glycopeptides of SEQ ID NO: 1 on Rink resin to produce the C-terminal amides. Acetate removal from the glycosides was accomplished “on resin” with hydrazine hydrate (H₂N—NH₂·H₂O) per previously reported methods. See, for example, Li et al., J. Med. Chem. 2014, 57, 2237-2246. doi: 10.1021/jm400879w. Three distinct coupling methods were used to assemble the glycopeptide backbone that contains two “difficult sequences” that can form aspartimides leading to α to β amide migration. This complication was avoided by the use of dipeptides with pre-formed amide linkages to each aspartic acid residue.

General: Peptide synthesis was performed on a Prelude® automated peptide synthesizer. Synthesis was performed either in an automated fashion or semi-manually where reagents were loaded into the reaction vessels using a syringe. The resin was agitated (mixed) using a steady flow of argon. The washing steps with DMF and DCM were performed for 2 minutes each.

Rink Amide Resin Preparation: 0.25 mmol of Rink Amide-MBHA resin (0.6 g) resin was placed in a 45 mL reaction vessel and swelled in DMF for 1 hour. Fmoc removal was achieved by addition of a solution containing 2% DBU-3% piperidine in DMF (6 mL) and mixing for 4 minutes. The mixture was then drained, and the resin was washed once with 6 mL of DMF. Fmoc removal was then repeated for an additional 8 minutes followed by 6 DMF washes (6 mL, 2 minutes).

Serine or Glycosyl Amino Acid Loading: 0.20 mmol, (0.8 eq.) of the desired first amino acid (Fmoc-Ser(tBu)—OH, Fmoc-Ser(Glc(OAc)₄)—OH, or Fmoc-Ser(Lac(OAc)₇)—OH) and 0.2 mmol, (0.8 eq.) 6-Cl-HOBt were placed into a vial and dissolved in 4 mL NMP. 0.2 mmol (0.8 eq.) of DIC was then added into the solution. The mixture was vortexed and/or sonicated for 1 minute and then added to the resin. The reaction mixture was mixed overnight for 16 hours. The mixture was diluted with DMF (10 mL) and drained immediately. Then the resin was washed 6 times with DMF (6 mL) and then 6 times with DCM (6 mL). The unreacted NH₂ sites on the resin were then capped with a solution of 10% N,N-diisopropylethylamine and 10% Ac₂O in 8 mL DCM This reaction was allowed to proceed for 1 hour. The resin was then washed 6 times with DCM (6 mL) and then washed 4 times with DMF (6 mL) to prepare the resin for the next automated steps.

Loading of Additional Fmoc-Ser(Glc(OAc)₄)—OH (CRA_DV3024 only): 0.5 mmol, (2.0 eq.) of Fmoc-Ser(Glc(OAc)₄)—OH and 0.5 mmol, (2.0 eq.) 6-Cl-HOBt were placed into a vial and dissolved in 4 mL NMP. 0.5 mmol (2.0 eq.) of DIC was then added into the solution. The mixture was vortexed and/or sonicated for 1 minute and then added to the resin. The reaction mixture was mixed overnight for 16 hours. The mixture was diluted with DMF (10 mL) and drained immediately. Then the resin was then washed 6 times with DMF (6 mL).

Prelude® Automated Synthesis: The Leu²³-Tyr¹⁰ amino acid series was prepared using the automated SPPS feature on the Prelude® automated peptide synthesizer. The Fmoc group was removed as described above and a solution containing the desired Fmoc-amino acid (2 equivalents). HBTU (2 equivalents), and N-methylmorpholine (10 equivalents) was loaded to the resin. The reaction mixture was mixed for 30 minutes followed by a single DMF wash (6 mL). The coupling reaction was repeated a second time for 30 minutes, and the resin was then washed 6 times with DMF (6 mL). Subsequent deprotection and coupling cycles were then performed up to Tyrosine¹⁰.

Manual Loading of DS Dipeptide: The Fmoc group was initially removed as described above. Then, 0.3 mmol of Fmoc-DS-OH or Fmoc-DG-OH (1.2 equiv.) and 0.3 mmol of 6-Cl-HOBt (1.2 equiv.) were added to a vial and dissolved in 4 mL of NMP. 0.3 mmol of DIC (1.2 equiv.) was then added to the solution. The mixture was vortexed and/or sonicated for 1 minute and then added to the resin. The reaction mixture was mixed for 16 hours. The mixture was diluted with DMF (10 mL) and drained immediately. Then the resin was washed 6 times with DMF (6 mL).

Automated Addition of IFT: The Ile⁵-Thr⁷ amino acid series was prepared using the automated SPPS feature on the Prelude® automated peptide synthesizer. The Fmoc group was removed as described above and a solution containing the desired Fmoc-amino acid (4 equivalents), HBTU (4 equivalents), and N-methylmorpholine (16 equivalents) in 10 mL of DMF was loaded to the resin. The reaction mixture was mixed for 30 minutes followed by a single DMF wash (10 mL) The coupling reaction was repeated a second time for 30 minutes, and the resin was then washed 6 times with DMF. Subsequent deprotection and coupling cycles were then performed up to Isoleucine⁵.

Manual Loading of DG Dipeptide: The Fmoc group was initially removed as described above. Then, 0.5 mmol Fmoc-DG-OH (2.0 equiv.) and 0.5 mmol of 6-Cl-HOBt (2.0 equiv.) were added to a vial and dissolved in 4 mL of NMP. 0.5 mmol of DIC (2.0 equiv.) was then added to the solution. The mixture was vortexed and/or sonicated for 1 minute and then added to the resin. The reaction mixture was then mixed for 6 hours. The mixture was diluted with DMF (10 mL) and drained immediately. Then the resin was washed 6 times with DMF (6 ml).

Manual loading of Fmoc-Ala-OH (CRA_TG_3026) or Fmoc-Sar-OH (CRA_TG_3027): The Fmoc group was initially removed as described above. Then, 1.0 mmol of amino acid (4 equiv.) and 1.0 mmol of 6-Cl-HOBt (4 equiv.) were added to a vial and dissolved in 4 ml of NMP. 1.0 mmol of DIC (4 equiv.) was then added to the solution. The mixture was vortexed for 1 minute and then added to the resin. The reaction mixture was mixed for 2 hours. The mixture was diluted with DMF (10 mL) and drained immediately. Then the resin was washed 6 times with DMF (6 mL).

Manual loading of remaining amino acids Fmoc-Asp(IBu)—OH (CRA_TG_3026 and CRA_TG_3027 only). Fmoc-Ser(tBu)—OH, and Fmoc-His(Trt)-OH: The Fmoc group was initially removed as described above. Then, 1.0 mmol amino acid (4 equiv.) and 1.0 mmol of 6-Cl-HOBt (4 equiv.) were added to a vial and dissolved in 4 mL of NMP. 1.0 mmol of DIC (4 equiv.) was then added to the solution. The mixture was vortexed for 1 minute and then added to the resin. The reaction mixture was mixed for 60 minutes. The mixture was diluted with DMF (10 mL) and drained immediately. Then the resin was washed 6 times with DMF (6 mL). After the final amino acid the Fmoc group was initially removed as described above.

Acetyl Cleavage: 120 mL of a 50% solution containing NH₂NH₂×H₂O in NMP (10 mL per reaction vessel) was prepared and added to the resin. The solution was mixed overnight for 16 hours. The solution was then drained, and a second 10 mL portion of 50% NH₂NH₂×H₂O was added to each reaction vessel. This solution was mixed for an additional 2 hours. The 50% NH₂NH₂×H₂O was then drained and the resin was washed 8 times with DMF (10 mL), 8 times with DCM (10 mL), and dried under vacuum for 3 hours.

Cleavage from the Resin and Global Side Chain Deprotection: The dried resin was treated with an acidic cleavage cocktail containing TFA, DCM, H₂O, triethylsilane, and anisole (90:10:2:3:0.5). The resin was mixed for 1 hour, and the solution was collected into a 45 mL centrifuge tube. The cleavage step was repeated 2 more times for 10-minute periods. The combined fractions were slowly evaporated over a stream of argon until the peptide began to crash out. Cold ether (about 40 mL) was then added to precipitate the peptide and the mixture was centrifuged for 10 minutes at 5 G. The ether layer was decanted off and ether (˜40 mL) was added to the crude peptide and centrifuged once more. This process was repeated for a third time. After decanting the ether layer, the crude peptide was dried under vacuum overnight.

HPLC Purification and Characterization of Peptides: These crude samples were then purified on a Gilson system with a UV detector (at 280 nm) using a Vydak C18 preparative reversed-phase column (250 mm×50 mm) using a gradient of 5-80% CH₃CN vs 0.1% CF₃COOH in H₂O over 60 min to give the glycopeptides in pure form, assessed for purity by analytical HPLC (Inspire C18 5 μm 250 mm×4.6 mm column) on a Varian LC with a diode array detector system (at 280 nm) employing the same gradient over a period of 25 min.

The pure fractions obtained from preparative HPLC purification were frozen at −80° C. and then lyophilized to afford the pure peptides as white and fluffy solids. The pure peptides were then characterized using mass spectrometry (ESI-MS).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges, or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A composition comprising a glycopeptide, said glycopeptide comprising a peptide that is covalently linked to a saccharide, wherein said peptide consists of from 20 to 25 amino acid residues and at least 75% sequence identity to SEQ ID NO: 1:
HSDX¹IFTDSYSRYRKQX²AVKKYLX³X⁴ (SEQ ID NO:1)
wherein
X¹ is glycine, alanine, sarcosine, valine, leucine, isoleucine, alpha-amino-isobutyric acid, or other aliphatic amino acids;
X² is norvaline, methionine, leucine, isoleucine, alpha-amino-isobutyric acid, or other aliphatic amino acids;
X³ is serine, threonine, cysteine, or an allo-threonine or a related compound; and
X⁴ can be absent or is serine, threonine, cysteine, or an allo-threonine or a related compound,
and wherein said glycopeptide comprises from 1 to 8 covalently linked carbohydrates.

2. The composition of claim 1, wherein at least one serine is glycosylated.

3. The composition of claim 1, wherein the C-terminus end of said peptide comprises two glycosylated amino acids.

4. The composition of claim 1, wherein at least one amino acid residue is a (D)-isomer.

5. The composition of claim 1, wherein X² is norvaline.

6. The composition of claim 1, wherein X³ is glycosylated with a mono- or a di-saccharide.

7. The composition of claim 1, wherein the sequence of the peptide is selected from the group consisting of SEQ ID Nos: 2-7.

8. The composition of claim 1, wherein X⁴ is (L)-serine, (D)-serine, (L)-threonine, (D)-theronine, (L)-cysteine, or (D)-cysteine.

9. The composition of claim 8, wherein X⁴ is glycosylated with a mono- or a di-saccharide.

10. The composition of claim 1, wherein said glycopeptide is a PAC1 agonist.

11. The composition of claim 10, wherein PAC1 effective concentration (EC₅₀) of said glycopeptide is about 10 nM or less.

12. The composition of claim 1, wherein said glycopeptide shows additional binding and activation toward VPAC1

13. The composition of claim 1, wherein VPAC1 effective concentration (EC₅₀) of said glycopeptide is about 100 nM or lower.

14. The composition of claim 1, wherein said glycopeptide has a much lower binding affinity to VPAC2 compared to binding affinity to VPAC2 by a native PACAP₁₋₂₇.

15. The composition of claim 14, wherein said glycopeptide has at least 10× lower binding affinity to VPAC2 compared to said native PACAP₁₋₂₇.

16. The composition of claim 14, wherein VPAC2 effective concentration (EC₅₀) of said glycopeptide is about 1 μM or greater.

17. The composition of claim 1, wherein said saccharide comprises from 1 to 8 carbohydrates.

18. The composition of claim 1, wherein said saccharide is a monosaccharide, a disaccharide, or a combination thereof.

19. The composition of claim 1, wherein said peptide comprises a plurality of glycosylated amino acid residues.

20. The composition of claim 1, wherein said saccharide is selected from the group consisting of glucose, maltose, lactose, melibiose, maltotriose, sucrose, trehalose, altose, saccharose, maltose, cellobiose, gentibiose, isomaltose, primeveose, galactose, xylose, mannose, manosaminic acid, fucose, GalNAc, GlcNAc, idose, iduronic acid, glucuronic acid, sialic acid, and polysaccharides related to the Thompsen-Friedrich antigens (Tn), as well as gangliosides or globosides.

21. A method for treating a neurodegenerative disease in a subject, said method comprising administering to the subject in need of such a treatment a therapeutically effective amount of the composition of claim 1.

22. The method of claim 21, wherein said neurodegenerative disease is selected from the group consisting of amyotrophic lateral sclerosis, Huntington's Disease, Parkinson's Disease, migraine attacks, traumatic brain injury, stroke, and dementia.


SEQUENCE LISTING (7 sequences)

SEQ ID NO: 1 - protein, 25 aa, Artificial Sequence
  synthetic
  misc_feature (4)..(4): glycine, alanine, sarcosine, valine, leucine, isoleucine, alpha-amino-isobutyric acid, or other aliphatic amino acids
  misc_feature (17)..(17): norvaline, methionine, leucine, isoleucine, alpha-amino-isobutyric acid, or other aliphatic amino acids
  misc_feature (24)..(24): serine, threonine, cysteine, or an allo-threonine or a related compound
  misc_feature (25)..(25): absent or serine, threonine, cysteine, or an allo-threonine or a related compound
HSDXIFTDSYSRYRKQXAVKKYLXX

SEQ ID NO: 2 - protein, 24 aa, Artificial Sequence
  Synthetic
  misc_feature (17)..(17): Xaa is norvaline
HSDGIFTDSYSRYRKQXAVKKYLS

SEQ ID NO: 3 - protein, 24 aa, Artificial Sequence
  Synthetic
  misc_feature (17)..(17): Xaa is norvaline
  misc_feature (24)..(24): glycosylated by glucose
HSDGIFTDSYSRYRKQXAVKKYLS

SEQ ID NO: 4 - protein, 25 aa, Artificial Sequence
  Synthetic
  misc_feature (17)..(17): Xaa is norvaline
  misc_feature (24)..(25): glycosylated by glucose
HSDGIFTDSYSRYRKQXAVKKYLSS

SEQ ID NO: 5 - protein, 24 aa, Artificial Sequence
  Synthetic
  misc_feature (17)..(17): Xaa is norvaline
  misc_feature (24)..(24): glycosylated by lactose
HSDGIFTDSYSRYRKQXAVKKYLS

SEQ ID NO: 6 - protein, 24 aa, Artificial Sequence
  Synthetic
  misc_feature (17)..(17): Xaa is norvaline
  misc_feature (24)..(24): glycosylated by lactose
HSDAIFTDSYSRYRKQXAVKKYLS

SEQ ID NO: 7 - protein, 24 aa, Artificial Sequence
  Synthetic
  misc_feature (4)..(4): Xaa is Sarcosine
  misc_feature (17)..(17): Xaa is norvaline
  misc_feature (24)..(24): glycosylated by lactose
HSDXIFTDSYSRYRKQXAVKKYLS